(19) United States
(12) Patent Application Publication
(10) Pub. No.: US 2021/0039877 A1
(43) Pub. Date: Feb. 11, 2021

(54) FLUID DISPENSER DEVICE

(71) Applicant: APTAR FRANCE SAS, Le Neubourg (FR)

(72) Inventors: Christophe JACUK, Le Vaudreuil (FR); Gérard PAPET, Sotteville Les Rouen (FR)

(73) Assignee: APTAR FRANCE SAS, Le Neubourg (FR)

(21) Appl. No.: 17/015,241

(22) Filed: Sep. 9, 2020

Related U.S. Application Data
(63) Continuation of application No. 15/825,580, filed on Nov. 29, 2017, now abandoned.
Application No. 15/825,580 is a continuation of application No. 13/399,385, filed on Feb. 17, 2012, now abandoned.
(60) Provisional application No. 61/452,766, filed on Mar. 15, 2011.

(30) Foreign Application Priority Data
Feb. 17, 2011 (FR) FR-11.51287

(51) Int. Cl. B65D 83/54; B65D 83/38

(52) U.S. Cl. CPC B65D 83/54; A61M 15/009; B65D 83/38

(57) ABSTRACT

A fluid dispenser device comprising: a body (100) that is provided with a dispenser orifice (110); a reservoir (1) containing fluid and a propellant gas; and a metering valve (20) that is assembled on said reservoir (1); said reservoir (1) being movable in said body (100) so as to actuate the metering valve (20) and dispense a dose of fluid through said dispenser orifice (110), said metering valve (20) including a valve member (30) that slides in said metering valve (20) during actuation; said device further comprising at least one sealing element (40, 41, 42) so as to form a leaktight seal, at least one sealing element (40, 41, 42) of said device comprising COC elastomer.

### RELATED APPLICATION

This application is a continuation of pending U.S. application Ser. No. 15/825,580, filed Nov. 29, 2017, which is a continuation of U.S. application Ser. No. 13/399,385, filed Feb. 17, 2012 (abandoned), which claims the benefit under 35 U.S.C. § 119(e) of U.S. provisional patent application Ser. No. 61/452,766, filed Mar. 15, 2011 (expired), and priority under 35 U.S.C. § 119(a)-(d) of French patent application No. FR-11.51287, filed Feb. 17, 2011, the entire contents of all of which are incorporated herein by reference.

### BACKGROUND OF THE INVENTION

The present invention relates to a fluid dispenser device.

More particularly, the present invention relates to a pharmaceutical-fluid dispenser device of the metered dose inhaler type, commonly known as a pressurized Metered Dose Inhaler (pMDI). In that type of device, the pharmaceutical fluid that contains one or more active substances is associated with a propellant gas and is disposed in a reservoir under pressure. A metering valve is assembled on the reservoir and is actuated so as to dispense a dose of fluid on each actuation. Inhalers of that type comprise an outer body in which the reservoir may slide, generally axially, so as to actuate the valve and dispense the dose of fluid through a dispenser orifice, generally a mouthpiece. That type of device is well known in the state of the art. Numerous types of metering valves exist that may be used with that type of inhaler. In general, a metering valve comprises a valve body in which a valve member slides. The valve body contains a metering chamber, and when the valve member is pushed in, the metering chamber empties through said valve member under the effect of the propellant gas. When the valve member then returns to its rest position, a new dose is loaded into the metering chamber. In known manner, that type of pMDI includes one or more sealing elements. The sealing elements provide sealing at different locations, and, in conventional manner, there generally exists a neck gasket that is interposed between the metering valve, the reservoir, and the fastener ring that serves to fasten the valve on the reservoir. In addition, the valve itself includes one or more sealing gaskets that co-operate with the valve member when said valve member is at rest and/or when it moves towards its actuated position. In more common valves, the valve generally includes two gaskets known as “internal gaskets” against which the valve member slides in leaktight manner during actuation. The various sealing elements are thus likely to be in contact with the active substance contained in the fluid to be dispensed. They are also in contact with the propellant gas. Generally, the sealing elements are made out of material of the ethylene-propylene terpolymer rubber (EPDM), nitrile rubber, or chloroprene rubber, etc. type. All of those materials perform well to a greater or lesser extent depending on the properties under consideration, and they all present certain drawbacks. In particular, they are likely to interact with the active substance and/or with the propellant gas. It is thus desirable to find materials for making such sealing elements that interact as little as possible with said active substance and/or with said propellant gas, while being easy to manufacture and to assemble, so as to be suitable for typical high-speed assembly lines for such inhalers.

Document WO 98/07768 describes a material known as Cyclo Olefin Copolymer (COC) elastomer that has been developed in particular to make hoses, tubes, and flexible pouches in the medical field. However, that material has never been used in devices of the inhaler type, and in particular in contact with propellant gases that act very aggressively on the component materials of sealing elements. However, it has been observed, surprisingly, that COC elastomer material turns out to be particularly beneficial and suitable for being used in metering valve applications in which said valve functions with a propellant gas, in particular of the hydrofluoroalkane (HFA) type.

### SUMMARY OF THE INVENTION

An object of the present invention is thus to provide a fluid dispenser device that does not have the above-mentioned drawbacks.

More particularly, an object of the present invention is to provide a fluid dispenser device that improves the properties of the sealing elements used in the device, and that limits the damaging interactions between said sealing elements and the fluid and/or the propellant gas with which it is in contact.

Another object of the present invention is to provide a fluid dispenser device that is simple and inexpensive to manufacture and to assemble.

The present invention thus provides a fluid dispenser device comprising: a body that is provided with a dispenser orifice; a reservoir containing fluid and a propellant gas; and a metering valve that is assembled on said reservoir; said reservoir being movable in said body so as to actuate the metering valve and dispense a dose of fluid through said dispenser orifice, said metering valve including a valve member that slides in said metering valve during actuation; said device further comprising at least one sealing element so as to form a leaktight seal, at least one sealing element of said device comprising COC elastomer.

Advantageously, said metering valve is assembled on said reservoir with a neck gasket interposed therebetween.

Advantageously, said metering valve includes at least one internal gasket that co-operates in leaktight manner with said valve member.

Advantageously, said metering valve includes an upper internal gasket and a lower internal gasket, defining between them a metering chamber of said metering valve.

Advantageously, said neck gasket and/or said upper internal gasket and/or said lower internal gasket comprise(s) COC elastomer.

Advantageously, said at least one sealing element is constituted by COC elastomer.

Advantageously, said fluid is a pharmaceutical fluid containing at least one active substance. Advantageously, said propellant gas comprises HFA gases of the HFA 134a and/or HFA 227 type.

Advantageously, a ring is associated with the metering valve, at least one sealing element made of COC elastomer being over-molded on a portion of said metering valve and/or of said ring.

Advantageously, COC elastomer is an elastomeric copolymer having a glass transition temperature between −10° C. and +15° C., a crystalline melting temperature between 50° C. and 120° C., a crystallinity by weight between 5% and 40% and a norbornene content between 2 and 15 mol %.

These advantages and others of the present invention appear more clearly from the following detailed description of an advantageous embodiment thereof, given by way of non-limiting example, and with reference to the accompanying drawing.

### BRIEF DESCRIPTION OF THE DRAWINGS

FIG. 1 is a diagrammatic section view of a fluid dispenser device of the Metered Dose Inhaler (MDI) type;

FIG. 2 is a diagrammatic section view of a reservoir on which a metering valve is assembled, in an advantageous embodiment of the present invention; and

FIG. 3 is a chart showing extractable level for COC elastomer compared to the TPE.

### DETAILED DESCRIPTION OF THE INVENTION

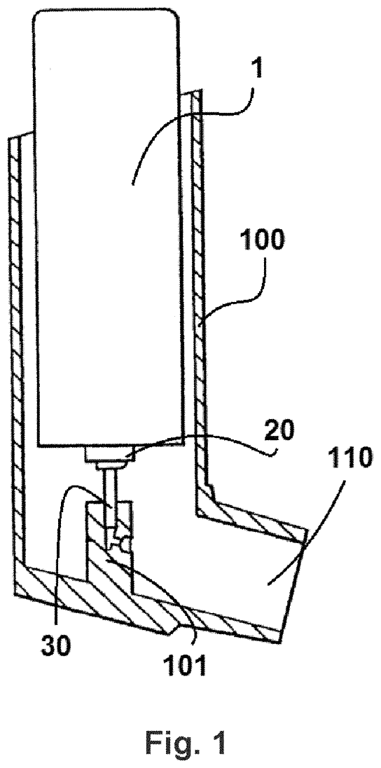

With reference to FIG. 1, there is described a metered dose inhaler, generally known as a pMDI, that conventionally includes an outer body 100 provided with a dispenser orifice 110, generally a mouthpiece. Inside the body there is disposed a reservoir 1 on which a metering valve 20 is mounted. A valve member 30 slides in said metering valve 20 so as to dispense a dose of fluid on each actuation. The body 100 includes a well 101 that receives the valve member 30, and that creates a connection passage between the outlet of the valve member 30 and said dispenser orifice 110. In conventional manner, in order to actuate such a device, the user presses on the end of the reservoir 1 so as to push said reservoir axially inside the body 100, thereby causing the valve member 30 to slide in leaktight manner into the metering valve 20, thereby causing a dose of fluid to be dispensed. Inside the reservoir, the fluid, which generally contains one or more active substances, is associated with a propellant gas, preferably a gas of the HFA type, e.g. HFA 134a and/or HFA 227.

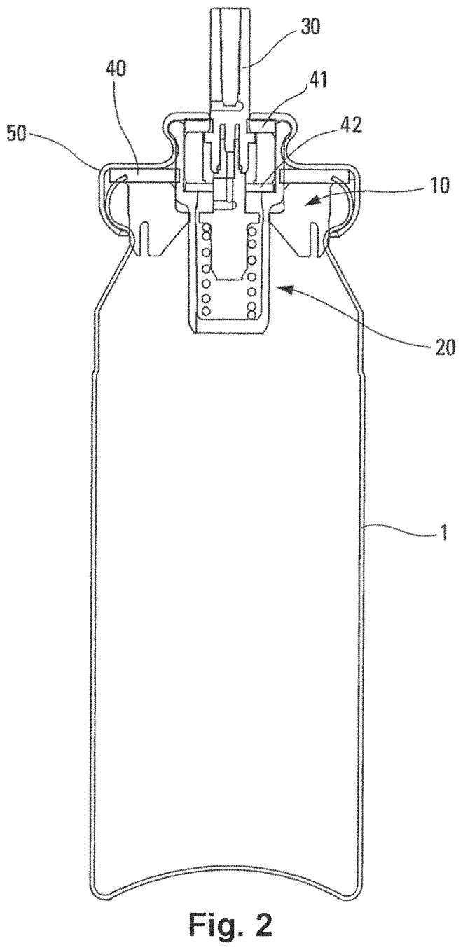

FIG. 2 shows a metering valve in a particular advantageous embodiment. Naturally, the present invention is not limited to that type of metering valve, but applies to any type of metering valve that can be used in pMDIs. A metering valve 20 is thus assembled on the reservoir 1, as can be seen in FIG. 2. Assembly may be achieved by means of a fastener ring 50 that, in this configuration, is a ring fastened by crimping, but that could also be a ring that is snap-fastened or screw-fastened. The metering valve 20 conventionally comprises a metering valve inside which a valve member 30 slides. The valve member 30 is urged by a spring towards its rest position. In known manner, a sealing gasket known as a “neck gasket” 40 is interposed between the fastener ring 50 and the neck of the reservoir 1 while the metering valve 20 is being assembled on the reservoir 1, so as to provided sealing at the neck of the reservoir. In addition, the metering valve includes at least one, and in this configuration two, internal sealing gaskets 41, 42 that co-operate in leaktight manner with the valve member 30. Thus, as shown in FIG. 2, the valve includes an upper internal gasket 41 and a lower internal gasket 42, the terms “lower” and “upper” referring to the orientation in FIG. 2, i.e. with the valve 20 disposed above the reservoir 1. A metering chamber is defined between the two internal gaskets 41, 42, and when the valve member 30 is pushed into the valve, the contents of the metering chamber are expelled through the valve member in conventional manner. A ring 10 may be interposed between the neck gasket and the valve body so as to limit contact between the active substance and the neck gasket 40, but also so as to limit the dead volume in this location of the device. When present, the ring may be of any appropriate shape and material.

In the invention, at least one of the sealing elements, i.e. at least one of the neck gasket 40, the upper internal gasket 41, and the lower internal gasket 42, comprise COC elastomer. Preferably, the three above-mentioned gaskets are made of that material. Advantageously, COC elastomer forms the only base material, but it is possible to envisage making a COC elastomer alloy with one or more other materials, in particular of the elastomer type.

COC elastomer is manufactured and sold by the supplier TOPAS, in particular.

COC is a copolymer that is formulated with a norbornene ring and polyethylene. Norbornene comes from synthesizing ethylene and a cyclopentadiene. Typical COC thus is a substantially rigid material. COC elastomer is thus a COC in which the polyethylene content is greater, thereby imparting elastomeric properties to said material. COC elastomer thus is not a mixture or a blend of typical COC with an elastomeric material, but is itself a material having some properties similar to elastomeric materials.

COC elastomer is a material having a glass transition temperature between −10° C. and +15° C., a crystalline melting temperature between 50° C. and 120° C., a crystallinity by weight between 5% and 40% and a norbornene content between 2 and 15 mol %.

The advantages of COC elastomer are numerous.

Firstly, it presents a chemical nature that is very inert since, in contrast to other elastomer materials, it does not include any reactive open or available double bond.

COC elastomer also has a very low level of extractables, i.e. very few particles known as extractables leach out from gaskets made out of COC elastomer, even when the gaskets are in contact with HFA-type propellant gases that are particularly aggressive. In particular, COC elastomer is not having fatty acids as extractables, at the opposite of thermoplastic elastomers or elastomeric materials. The extractables existing with COC elastomer thus mainly comprise antioxidants.

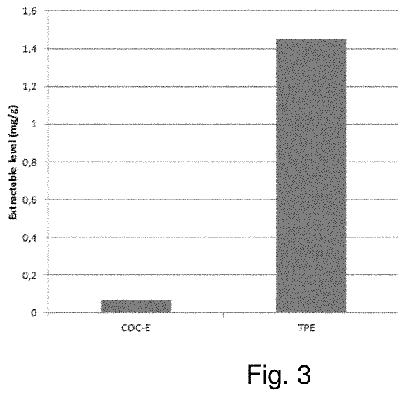

The chart in FIG. 3 proves that the extractable level is substantially lower for COC elastomer compared to the TPE (thermoplastic elastomer, which in this comparison is formed by the blend of 50% butyl and 50% polyethylene.

Elastomeric materials have even much higher extractable levels, as e.g. nitrile which has an extractable level of about 14 mg/g, or EPDM which has an extractable level between 1.4 and 5.3 mg/g.

COC elastomer also presents significant barrier properties against water vapor, and mechanical properties that are entirely suitable for making valve gaskets, in particular its hardness and its Young's modulus. In particular, it provides sealing performances (static leakage of propellant, moisture ingress) similar to TPE (thermoplastic elastomer) materials, such as the TPE described above (50% butyl and 50% polyethylene), and better performances than elastomers, e.g. EPDM.

It also presents the ability to withstand abrasion, and is capable of being molded cohesively on other polymers of the polyolefin type. In particular, a gasket made of COC elastomer may be molded on a portion of the valve and/or a portion of the ring 10, in particular when said valve and/or ring is/are made of a material having the same chemical nature, such as COC. COC elastomer also presents good compatibility with active substances of the pharmaceutical type since there is no leaching of ions, no trace metals, it includes hydrophobic surfaces so that there is less absorption, and finally it can be designed easily and flexibly, i.e. it is easy to make gaskets of any shape from this material.

By way of example and in non-limiting manner, the COC-E X1 T6 product sold by the supplier TOPAS ADVANCED POLYMERS is a material that is suitable for the present invention.

Surprisingly, it turns out that this material improves the operation of metering valves, reduces interactions between the material and the active substance and/or the propellant gas, and makes the manufacture and the assembly of valves and of inhalers in which the valves are used less difficult or less complicated, and thus less costly.

Although the present invention is described above with reference to an advantageous embodiment thereof, it is naturally not limited thereto, and any useful modifications could be applied thereto without going beyond the ambit of the present invention, as defined by the accompanying claims.

1. A sealing member for a dispenser device configured to contact a hydrofluoroalkane (HFA) propellant gas, the sealing member has an annulus shape and has a composition comprising:
a cyclo olefin copolymer elastomer including:
a norbornene ring; and
polyethylene,
wherein the cyclo olefin copolymer elastomer has a glass transition temperature of −10° C. to 15° C.

2. The sealing member according to claim 1, wherein an amount of the polyethylene in the cyclo olefin copolymer is sufficient to impart elastomeric properties to the sealing member.

3. The sealing member according to claim 1, wherein the cyclo olefin copolymer elastomer has a crystalline melting temperature of 50° C. to 120° C.

4. The sealing member according to claim 1, wherein the cyclo olefin copolymer elastomer has a crystallinity by weight of 5% to 40%.

5. The sealing member according to claim 1, wherein the nobornene content is 2 mol % to 15 mol %.

6. A fluid dispenser device comprising:
the sealing member according to claim 1
a body provided with a dispenser orifice;
a reservoir containing fluid and the HFA propellant gas; and
a metering valve assembled on the reservoir, the reservoir being movable in said body so as to actuate the metering valve and dispense a dose of fluid through the dispenser orifice, the metering valve comprising a valve member that slides in the metering valve during actuation.

7. The fluid dispenser device according to claim 6, wherein the sealing member is in direct contact with the HFA propellant gas.

8. The fluid dispenser device according to claim 7, wherein the sealing member forms a leaktight seal that seals against a sliding member of the metering valve.

9. The sealing member according to claim 1, wherein the cyclo olefin copolymer elastomer does not comprise fatty acid as extractables.

10. The sealing member according to claim 1, wherein the cyclo olefin copolymer elastomer includes hydrophobic surfaces.

11. The sealing member according to claim 1, wherein the cyclo olefin copolymer elastomer has an inert chemical nature without any reactive open or available double bond.

12. The sealing member according to claim 1, wherein the cyclo olefin copolymer elastomer is molded cohesively on other polyolefin polymers.

13. A fluid dispenser device comprising:
the sealing member according to claim 1
a body provided with a dispenser orifice;
a reservoir containing fluid and the HFA propellant gas and an active pharmaceutical substance; and
a metering valve assembled on the reservoir, the reservoir being movable in said body so as to actuate the metering valve and dispense a dose of fluid through the dispenser orifice, the metering valve comprising a valve member that slides in the metering valve during actuation;
wherein the metering valve is assembled on the reservoir by a fastener ring that is one of crimped, snap-fastened or screwed onto the reservoir;
wherein the sealing member is a neck gasket interposed between the fastener ring and a neck of the reservoir and in direct contact with the HFA propellant gas and the active pharmaceutical substance in the reservoir; and
wherein the fluid dispenser device further comprises two internal sealing gaskets that co-operate in a leaktight manner with the metering valve, and a metering chamber is defined between the two internal sealing gaskets such that the two internal sealing gaskets are in direct contact with the HFA propellant gas and the active pharmaceutical substance contained in the metering chamber during use of the dispenser device, each of the two internal sealing gaskets comprises a cyclo olefin copolymer elastomer including a norbornene ring and polyethylene, wherein the cyclo olefin copolymer elastomer has a glass transition temperature of −10° C. to 15° C.

14. The fluid dispenser device according to claim 13, wherein the HFA propellant gas is HFA 134a.

15. The fluid dispenser device according to claim 13, wherein the HFA propellant gas is HFA 227.

16. The fluid dispenser device according to claim 13, wherein the cyclo olefin copolymer elastomer of the sealing member and the cyclo olefin copolymer elastomer of the two internal sealing gaskets have a crystalline melting temperature between 50° C. and 120° C., a crystallinity by weight between 5% and 40% and a norbornene content between 2 and 15 mol %.

17. A fluid dispenser device according to claim 13, wherein the sealing member is a cylindrical annulus.

18. A fluid dispenser device according to claim 13, wherein each of the two internal sealing gaskets is a cylindrical annulus.
